(19) United States
(12) Patent Application Publication
(10) Pub. No.: US 2019/0015673 A1
(43) Pub. Date: Jan. 17, 2019

(54) METHOD AND APPARATUS FOR PREFERENTIALLY HEATING A SUBSTRUCTURE IN A COMPOSITE MATERIAL

(71) Applicant: MASSACHUSETTS INSTITUTE OF TECHNOLOGY, Cambridge, MA (US)

(72) Inventors: Admir Masic, Cambridge, MA (US); Karl D Brommer, Exeter, NH (US)

(73) Assignee: Massachusetts Institute of Technology, Cambridge, MA (US)

(21) Appl. No.: 16/068,292

(22) PCT Filed: Jan. 13, 2017
(86) PCT No.: PCT/US2017/013376

§ 371 Date: Jul. 5, 2018

Related U.S. Application Data
(60) Provisional application No. 62/278,516, filed on Jan. 14, 2016.

(51) Int. Cl. A61N 1/40; A61B 5/00; A61B 5/01

(52) U.S. Cl. CPC A61N 1/403; A61B 5/0075; A61B 2576/00; A61B 5/4836; A61B 5/015

(57) ABSTRACT

A system (2) for transferring radio frequency energy to a composite structure (22) is provided that includes a radio signal generator (14) that produces a pulse modulated waveform with variable carrier frequency and variable pulse repetition frequency. The pulse modulated waveform is applied to the composite structure (22) for heating. An infrared imaging element (4, 6) measures the rate of heating in the composite structure (22) for particular values of the variable carrier frequency and the variable pulse repetition frequency. The infrared imaging element (4, 6) produces a representation illustrating the effects of heating on the composite structure (22) as well as the composite's molecular, nanoscopic structural, or chemical characteristics. A controller (8) determines the optimum variable center frequency and the variable pulse repetition frequency for optimum heating of the composite structure (22) while minimizing damage.

### PRIORITY INFORMATION

This application claims priority from provisional application Ser. No. 62/278516 filed Jan. 14, 2016, which is incorporated herein by reference in its entirety.

### BACKGROUND OF THE INVENTION

The invention is related to the field of biological composite structures, and in particular to preferentially heating a substructure in a biological composite material.

With modern medical advances and improved quality of life, human life expectancy is constantly increasing. One is continually confronted with new sets of health-related problems (e.g. age-related pathologies such as osteoporosis and skin aging). Anti-aging skin care is a large and dynamic business, accounting for 15% of the global skin care market worth currently US$ 110 billion and, according to data from Euromonitor International, is growing at a 5% compound annual rate. Anti-aging products are expected to be key drivers of that growth.

The aging process occurs within all organs of the body, but manifests itself visibly in the skin. Cutaneous aging consists of processes that are a direct result of either intrinsic or extrinsic factors. Whereas intrinsic aging is a time-dependent process and is influenced by a person's genetic predispositions, extrinsic aging, which includes photoaging, is driven by environmental forces. The hallmark of photodamaged skin is the accumulation of elastin-containing fibrils in the dermis, a process known as elastosis. This process is accompanied by a decrease in collagen synthesis and architectural changes in the collagen fiber network, which becomes more disorganized. The most obvious clinical manifestations of aging skin are the increased formation of wrinkles, dyschromia, and skin laxity. From the macroscopic perspective, decreased water concentration in the superficial layers of the skin tissue is known to cause an observable alteration in the physical characteristics of the skin surface, which is noted objectively as dry and scaly skin. All these macroscopic detrimental changes to tissue properties can ultimately be attributed to alterations in the molecular and supra-molecular structure and chemistry of extracellular matrices including collagen and elastin. At the molecular level, physicochemical changes related to skin aging include increased intermolecular cross-linking and side-chain modifications of collagen and elastin, which are responsible for skin's tensile strength and elasticity, respectively.

A composite structure is defined as an agglomeration of a set of substructures. Examples include skin, cartilage, wood, concrete, plastics. Examples may be biological, organic, inorganic, natural or man-made.

### SUMMARY OF THE INVENTION

According to one aspect of the invention, there is provided a system for transferring radio frequency energy to a composite structure is provided. The system includes a radio signal generator that produces a pulse modulated waveform with variable carrier frequency and variable pulse repetition frequency. The pulse modulated waveform is applied to the composite structure for heating. An infrared imaging element measures the rate of heating in the composite structure for particular values of the variable carrier frequency and the variable pulse repetition frequency. The infrared imaging element produces a representation illustrating the effects of heating on the composite structure as well as the sample's molecular, nanoscopic structural, or chemical characteristics. A controller determines the optimum variable center frequency and the variable pulse repetition frequency for optimum heating of the composite structure while minimizing damage.

According to another aspect of the invention, there is provided a method of transferring radio frequency energy to a composite structure. The method includes producing a pulse modulated waveform with variable carrier frequency and variable pulse repetition frequency using a radio signal generator. The pulse modulated waveform is applied to the composite structure for heating. Also, method includes measuring the rate of heating in the composite structure for particular values of the variable carrier frequency and the variable pulse repetition frequency using an infrared imaging element. The infrared imaging element produces a representation illustrating the effects of heating on the composite structure as well as the sample's molecular, nanoscopic structural, or chemical characteristics. Furthermore, the method includes determining the optimum variable center frequency and the variable pulse repetition frequency for optimum heating of the composite structure while minimizing damage using a controller.

According to another aspect of the invention, there is provided a system for transferring radio frequency energy to a collagen structure. The system includes a radio signal generator that produces a pulse modulated waveform with variable carrier frequency and variable pulse repetition frequency. The pulse modulated waveform is applied to the collagen structure for heating. An infrared imaging element measures the rate of heating in the composite structure for particular values of the variable carrier frequency and the variable pulse repetition frequency. The infrared imaging element produces a representation illustrating the effects of heating on the collagen structure as well as the sample's molecular, nanoscopic structural, or chemical characteristics. A controller determines the optimum variable center frequency and the variable pulse repetition frequency for optimum heating of the collagen structure while minimizing damage.

### BRIEF DESCRIPTION OF THE DRAWINGS

FIG. 1 is a schematic diagram illustrating a system for implementing the invention;

FIG. 2 is a process flow graph illustrating the operations of a controller used in accordance with the invention;

FIG. 3 is a process flow graph illustrating the operations of a controller used in accordance with FIG. 2 using a Raman spectrometer;

FIG. 4 is a schematic diagram illustrating a Raman spectrum for a collagen and water mixture;

FIG. 5 is a process flow graph illustrating a drying process used in accordance with the invention;

FIG. 6 is a schematic diagram illustrating a RF generator used in accordance with the invention;

FIG. 7 is a schematic diagram illustrating the circuit representation of the RF generator used in FIG. 6; and

FIG. 8 is a schematic diagram illustrating a circuit having a cascaded arrangement to heat larger samples.

### DETAILED DESCRIPTION OF THE INVENTION

This invention pertains to the treatment of skin, specifically by transferring radio frequency energy to collagen. The technique applies to other human tissue, including bones, ligaments and tendons. The technique also applies to non-biological composites, including concrete and carbon composites. The invention utilizes electric fields at a specific radio frequency and waveform while monitoring the process with a Raman spectrometer.

By sweeping the electric signal through a range of center frequencies, pulse widths and pulse repetition frequencies, the heating can be tuned to a specific sub-structure in the composite while surrounding sub-structures are substantially less affected by the applied electric field. RF heating is commonly employed in skin treatment and other forms of cellular regeneration and healing therapies with FDA approval. However, heating effectiveness is limited by damage and pain caused in surrounding tissue because current radio waveforms are not tailored to the chemistry of the area under treatment. A Raman spectrometer provides closed loop feedback to monitor and control the heating process by directly observing which sub-structures in a composite material are most efficiently absorbing a particular radio waveform.

The inventive technique preferentially heats a specific sub-structure of interest embedded in the composite. This solution also applies to drying processes and preservation.

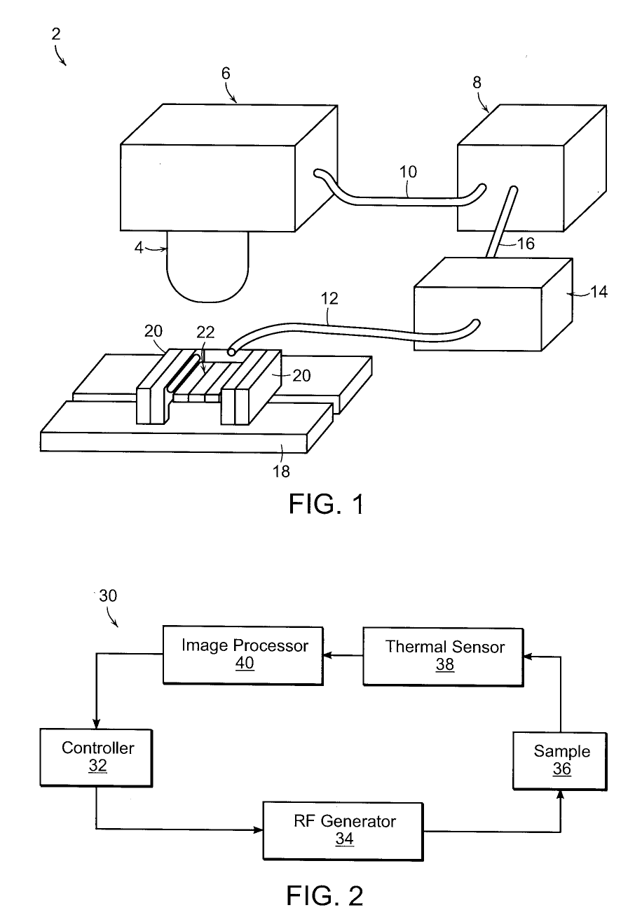

FIG. 1 is a schematic diagram illustrating a system 2 for implementing the invention. The system 2 includes a clamp 20 that secures samples 22 under an infrared imaging element 4, either a Raman spectrometer or a thermal imaging camera. The clamps 20 are situated in a toothed track for maintaining proper separation. For hard samples, the clamp 20 is neutral or in compression. For soft samples, the clamp 20 is in tension. The surfaces of the clamp jaws are coated with an electrically insulating material, for example Teflon or kapton tape. One surface of each clamp 20 has an electrically conductive layer on top of the insulation to be connected to the electrodes in a coaxial cable 12. The electrodes must be kept short, no more than 10 mm or so in order reduce stray capacitance of the RF signal coming out of the cable. The coaxial cable 12 connects to a radio signal generator 14 which is generating anywhere from a few hundred milliwatts to thousands of kilowatts depending on the material and the type of sample 22. For initial experimentation and development of RF heating waveforms, samples will be small and power levels will be low.

The radio signal generator 14 produces a pulse modulated waveform with variable carrier frequency and variable pulse repetition frequency while operating under control of a computer or possibly a human observer. An infrared imaging element 6 measures the rate of heating in the sample 22 for particular values of the carrier frequency and the pulse repetition frequency. The measurement is made in one of two ways. For a thermal camera, regions of the composite material must be identified containing the sub-element to be selectively heated. For many materials, magnification optics may be needed to enlarge the region of interest in the image recorded by the focal plane of the thermal camera. The image shows regions heating more quickly in a different color. Typically commercial thermal imagers show hotter regions in red and colder regions in blue as one would intuitively expect.

If the infrared imager 6 is a Raman spectrometer, different sub-elements of the composite will contain different molecules. Therefore, the relative intensities of spectral lines characteristic of molecules in the two regions will vary with their relative temperature. To determine the optimum waveform for selectively heating the sub-element of the composite of interest, a computer 8 sweeps through a set of candidate pairs {fc, fp) where fc denotes the center frequency and fp denotes the pulse repetition frequency, while observing the relative heating of sub-elements in the composite. The optimum values {fc*, fp*} are determined by the maximum difference in heating between the regions subject to the constraint of a minimum degree of heating of the sample between sub-elements of the composite. This system empirically determines the waveform in situ and can then proceed to dry the samples using the optimum waveform. Alternatively, the waveform can be incorporated in a specialized embodiment of the invention for mass production applications including industrial processes and medical therapies.

The computer 8 is coupled to the imager 6 via cable 10 and respectively transmits the necessary radio parameters that includes preferential heating parameters. The radio parameters are transmitted to the radio signal generator 14 via cable 16. The radio signal generator 14 uses the radio parameters to implement the desired heating of the sample 22.

FIG. 2 is a process flow graph 30 illustrating the operations of a controller 32 used in accordance with the invention. The controller 32 determines optimum carrier frequency and pulse repetition frequency {fc,fp} by sending candidate values in a control signal to the RF generator 34. The RF generator 34 heats a sample 36 according to the electrical waveform A₀ sin (2πf_{c}t+φ)p_{fp}(t) where A0 is the amplitude supplied by the generator and p_{fp}(t) denotes a pulse train at frequency fp. Typically the pulse train is a square wave at fifty per cent duty cycle. As the sample 36 is heated, a thermal camera 38 incorporating magnification and focus records the image on a digital focal plane to be transmitted to the image processor. Usually the image processor 40 is software running in a general-purpose processor, however, a graphics processor, an FPGA or other specialized hardware device may be possible. The image processor 40 determines the temperature of each sub-element in the images in two steps. First, it assigns focal plane pixels to specific composite sub-elements. Next it determines the temperature of each sub-element by averaging the intensities of all pixels imaging each sub-element.

Note that the first step of assigning pixels to each sub-element must only be done once at the beginning of the heating process provided that the optics and the sample 36 do not move. For each particular heating waveform, the image processor reports a set of temperatures {T0,T1, . . . } for all sub-elements in the composite with T0 denoting the sub-element to be heated preferentially. The control records the set {T} then may pause the heating to let the sample cool or the sample may cool automatically due to ambient thermal conduction depending on how the sample fixture conducts heat and how much power is supplied by the RF generator 34. Ideally one obviously wants to maintain the sample at a relatively constant temperature so that many candidate waveforms may be evaluated in the shortest possible time. The controller 32 continues to supply the RF generator 34 with candidate {fc,fp} and recording the resulting {T} over the entire set of possible {fc,fp}. After the last candidate waveform has been tested, the controller 34 determines optimum, {fc*,fp*} subject to one of two criteria: Either max {T0-Ti} subject to min T0 or max T0 subject to min {T0-Ti}.

The first case corresponds to samples where adequate RF heating is easy and relative heating must be minimized in order to prevent damage to collateral sub-elements. The second case corresponds to samples where RF heating is more difficult and a minimum amount of heat must be applied within the constraint of not collaterally damaging the sample.

It may be possible that neither criterion can be satisfied. In this case, an RF generator with more power may be needed, a smaller sample, or a different configuration of electrodes.

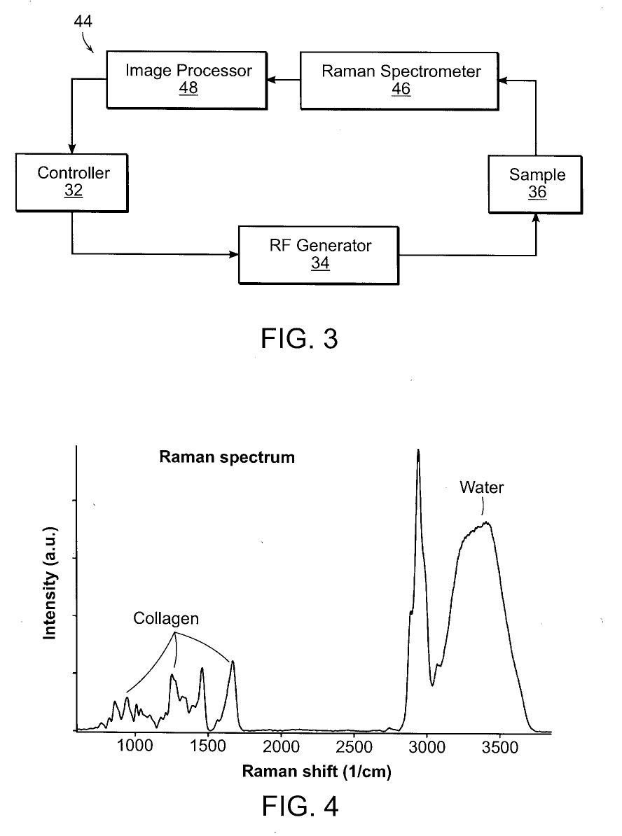

FIG. 3 shows a process flow 44 when a Raman spectrometer 46 replaces the thermal imager as shown in FIG. 2. In this case, laser excitation of the sample 30 provides the Raman spectral image, under magnification for samples with small sub-elements within the composite. The Raman spectrometer 46 detects optical spectral lines corresponding to vibrational and rotational states of the macromolecules and molecules characteristic of each sub-element in the composite. The Raman image processor 48 calculates temperatures of each sub-element using the spectral line intensities of vibrational states of each macromolecule. While the Raman spectrometer is a more complex instrument than the thermal imager 38, this approach has the advantages of avoiding more costly infrared optics and avoiding digital image processing to identify sub-elements in the composite.

FIG. 4 shows a Raman spectrum for a collagen and water mixture, one of the most important sub-elements in biological composites. It is important to note that the invention utilizes a radio waveform that makes the dipolar water molecules vibrate near the collagen, thereby heating it. The radio center frequency is chosen to make the water oscillate at its resonant piezoelectric frequency. The pulse repetition frequency is chosen at the mechanical resonant frequency of the sub-element, as determined by the apparatus shown in FIG. 1. The peak position, shape and intensity of the collagen spectrum indicates the degree of preferred heating for a particular waveform.

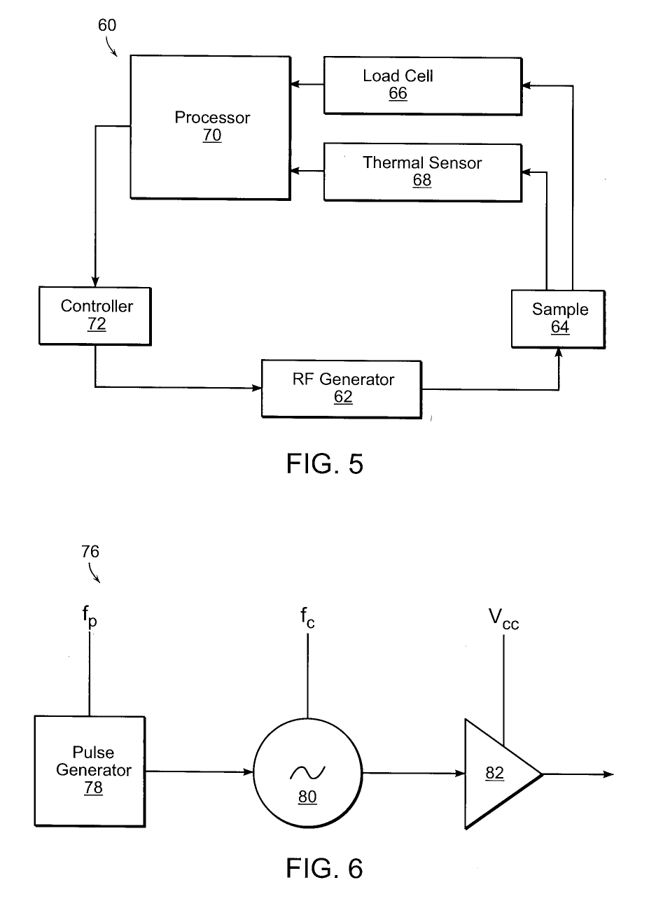

FIG. 5 shows a process flow 60 of a drying process used in accordance with the invention. The goal of the drying process is to remove water molecules from a sub-element of a composite through RF heating. In this case, as the RF generator 62 heats the sample 64, water molecules migrate away from macromolecules and other constituents of the sub-element and evaporate. As the sample 64 dries, tension increases between the clamps holding the sample 64 in place. This tension is measured by a load cell 66 attaches to the clamp holding the samples in tension and monitored by the processor 70 providing additional feedback on the rate of heating for the RF waveform, particularly the selected values for the center frequency and the pulse repetition frequency devised by the controller 72. The temperature experienced by the sample is measured by the thermal sensor 68 and sent to the processor 70 for monitoring and providing additional feedback on the rate of heating for the RF waveform.

FIG. 6 is a schematic diagram illustrating a RF generator 76 used in accordance with the invention. The RF generator 76 includes a pulse generator 78 that generates a square wave at frequency fp, typically in the kHz range, typically with 50% duty cycle, as input to an oscillator 80 set to oscillate the frequency fc, typically in the low MHz. An amplifier stage 82 amplifies the resulting radio pulse signal, with gain determined by the supply voltage of the amplifier. The resulting radio signal has the form A₀ sin (2πf_{c}t+φ)p_{fp}(t) where p_{fp}(t) is the pulse generator square wave at frequency fp, sin (2πf_{c}t+φ) is the oscillator signal at frequency fc, A is the amplifier output voltage determined by the supply voltage to the amplifier. Note how the pulse generator 78 feeds directly into the oscillator 80 without a mixer.

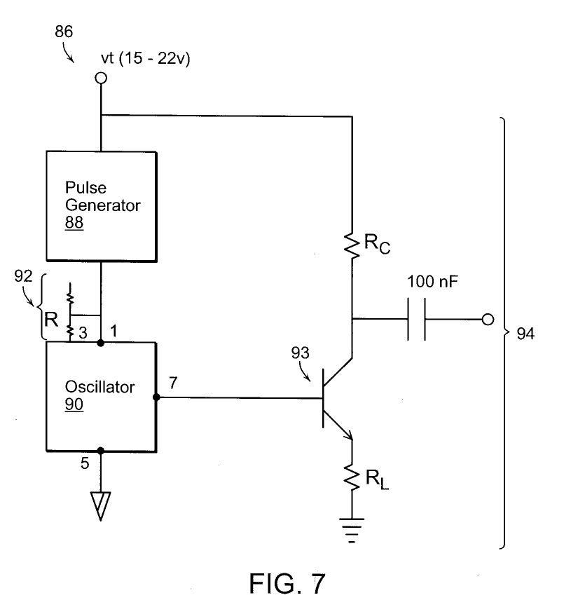

FIG. 7 is a schematic diagram illustrating the circuit representation of an RF generator 86 used in accordance with the invention. A pulse generator module 88 outputs a 0-5V square wave at a frequency in the kHz range as the power source to an oscillator module 90, in one embodiment, the LTC1799 sold by Linear Technology Inc. As long as the pulse repetition frequency is low compared to the carrier frequency, settling time of the oscillator 90 is negligible for a radiofrequency pulse intended to heat a sample. The resistor on pin 3 of the LTC1799 provides a voltage divider 92 to set the carrier frequency of the oscillator 90. The output on pin 7 is the radio pulse waveform, amplified by a Class C RF amplifier 94, in this case using a 2N3866 high-speed power transistor 93 or the like. Larger power transistors can be used to provide more output power at greater cost. The ratio of collector resistor Re and emitter resistor RL sets the gain as is well known. Rc also prevent burnout of the transistor and should be matched to the load impedance of mass production applications to a particular class of samples.

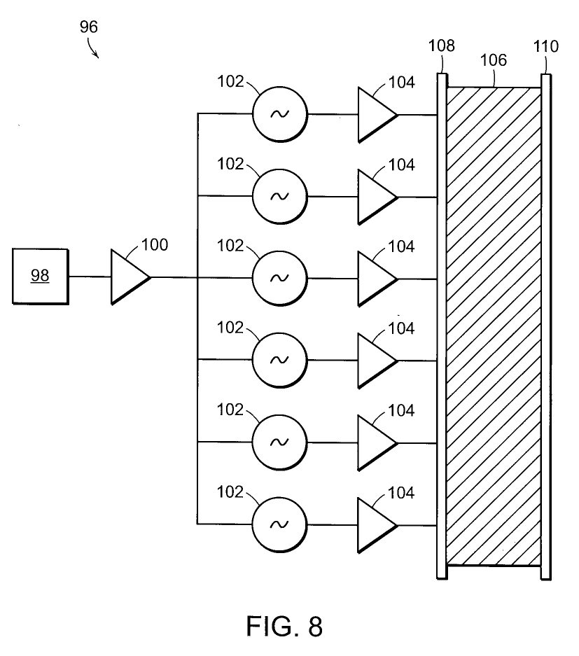

FIG. 8 shows how the circuit 96 in FIG. 7 can be cascaded to heat larger samples. A simple pulse generator 98 is amplified using an amplifier stage 100 to supply voltage and current for a bank of oscillators 102 similar to that shown in the previous figure. Each oscillator signal is amplified by an amplification stage 104 as before and connected to a large common electrode attached to the sample. Current flows from the electrodes 108 along a wide path throughout the samples 106 to a ground electrode 110 on the other side.

The invention attempts to locally image samples and skin areas under treatment in order to assess heating efficiency. It is anticipated that varying the center frequency of the RF waveform will change the interaction with water molecules and thereby change the effectiveness of energy transfer. It is anticipated that coherent RF waveforms can be more effective than incoherent radiation, also known as band-limited white noise. Pulses of varying repetition frequency and width to electromechanically stimulate the sample. Because tissue is ionic, it polarizes under application of an electric field. Mathematically, the tissue has an inhomogeneous, frequency-dependent dielectric function. It forms a piezo electric system and the specific form of the applied pulses will affect the degree and manner of interaction. As a mechanical system, the tissue contains normal modes of vibration.

The electrical waveforms are designed to optimally excite local normal modes. Because the dielectric function is frequency-dependent and inhomogeneous, a wave form optimized for local stimulation will heat areas outside the areas under treatment to a much lesser degree. For example, it is known from piezo beam mechanics that vibration resonances tend to have Q's around 100, meaning that coupling is only about one per cent outside the region of treatment compared to inside the region of treatment.

The RF pulse train, described above, is used to excite the normal vibration modes. By sweeping the center frequency, pulse repetition frequency and pulse width while observing efficiency with the spectrometers, one can identify the normal modes and choose optimum heating waveforms that efficiently couple to the area under treatment while remaining decoupled from other areas. This technique minimizes the amount of energy transmitted to the patient, thereby minimizing patient discomfort. Because skin and other tissue are inhomogeneous, the dielectric function is also inhomogeneous, the normal modes are inhomogeneous and local energy absorption occurs with this technique.

This invention uses coherent radio waveforms, the use of feedback in the form of Raman and infrared spectrometers with imaging techniques and the composition of the ionic gels for coupling the electrodes to the skin. Feedback using X-Ray spectroscopy is also possible.

In order to improve current treatment strategies and develop new and effective treatment options, it is crucial to understand the structural and chemical response of the skin matrix materials to external stimuli (such as temperature, RF, hydration, and specific mechanical, chemical, or topical treatment). This invention allows the exploration of precise structure-property relationships of skin extracellular matrix materials (i.e. collagen, elastin) including the molecular and nanoscopic structural and chemical characteristics associated with young and aged tissues and their response to external stimuli and treatments. Targeted anti-aging treatment strategies are developed through characterizing ex vivo and in situ the dynamic responses of collagen-based materials to external stimuli.

These objectives are realized by applying purely physical and chemical approaches based on in situ and multi-scale X-ray-Raman-mechanics experimental methodologies as well as advanced in situ, multi-scale and multi-spectral Raman-AFM-fluorescence chemical imaging techniques. The invention allows for the identification of links between chemistry, structure, and properties of skin extracellular matrix materials across multiple length scales and thus help lay the groundwork for the development of advanced diagnostic tools as well as more effective RF-based non-invasive skin rejuvenation strategies.

The invention can be relevant not only for anti-ageing strategies but potentially could impact many other fields where collagen-based materials are of crucial importance, including bone regeneration, wound healing, collagen-based biomaterials, fillers, scaffolds and creams, the leather and food industries. This invention also applies to composites not containing collagen, including concrete and carbon composites.

Although the present invention has been shown and described with respect to several preferred embodiments thereof, various changes, omissions and additions to the form and detail thereof, may be made therein, without departing from the spirit and scope of the invention.

What is claimed is:

1. A system for transferring radio frequency energy to a composite structure comprising:
a radio signal generator that produces a pulse modulated waveform with variable carrier frequency and variable pulse repetition frequency, the pulse modulated waveform is applied to the composite structure for heating;
an infrared imaging element that measures the rate of heating in the composite structure for particular values of the variable carrier frequency and the variable pulse repetition frequency, the infrared imaging element produces a representation illustrating the effects of heating on the composite structure as well as the sample's molecular, nanoscopic structural, or chemical characteristics; and
a controller that determines the optimum variable center frequency and the variable pulse repetition frequency for optimum heating of the composite structure while minimizing damage.

2. The system of claim 1, wherein the infrared imaging element comprises a thermal camera.

3. The system of claim 2, wherein the thermal camera illustrates regions of heating using color.

4. The system of claim 1, wherein the infrared imaging element comprises a Raman spectrometer.

5. The system of claim 4, wherein the Raman spectrometer illustrates position, shape and intensity of spectral lines characteristic of molecules.

6. The system of claim 1, wherein the controller determines the optimum carrier frequency and pulse repetition frequency by sending a control signal to the radio signal generator.

7. The system of claim 1, wherein the optimum carrier frequency and pulse repetition frequency are determined by the maximum difference in heating between regions subject to a constraint of a minimum degree of heating of the composite structure.

8. The system of claim 2, wherein the thermal camera incorporates magnification and focus to record an image on a digital focal plane to be transmitted to the image processor.

9. The system of claim 1, wherein the radio signal generator comprises a bank of oscillators that are coupled to a bank of amplifiers for amplification.

10. The system of claim 1, wherein the composite structure is a mixture of water and collagen.

11. The system of claim 10, wherein pulse modulated waveform vibrates water molecules near the collagen to produce heating.

12. A method of transferring radio frequency energy to a composite structure comprising:
producing a pulse modulated waveform with variable carrier frequency and variable pulse repetition frequency using a radio signal generator, the pulse modulated waveform is applied to the composite structure for heating;
measuring the rate of heating in the composite structure for particular values of the variable carrier frequency and the variable pulse repetition frequency using an infrared imaging element, the infrared imaging element produces a representation illustrating the effects of heating on the composite structure as well as the sample's molecular, nanoscopic structural, or chemical characteristics; and
determining the optimum variable center frequency and the variable pulse repetition frequency for optimum heating of the composite structure while minimizing damage using a controller.

13. The method of claim 12, wherein the infrared imaging element comprises a thermal camera.

14. The method of claim 14, wherein the thermal camera illustrates regions of heating using color.

15. The method of claim 12, wherein the infrared imaging element comprises a Raman spectrometer.

16. The method of claim 15, wherein the Raman spectrometer illustrates position, shape and intensity of spectral lines characteristic of molecules.

17. The method of claim 12, wherein the controller determines the optimum carrier frequency and pulse repetition frequency by sending a control signal to the radio signal generator.

18. The method of claim 12, wherein the optimum carrier frequency and pulse repetition frequency are determined by the maximum difference in heating between regions subject to a constraint of a minimum degree of heating of the composite structure.

19. The method of claim 13, wherein the thermal camera incorporates magnification and focus to record an image on a digital focal plane to be transmitted to the image processor.

20. The method of claim 12, wherein the radio signal generator comprises a bank of oscillators that are coupled to a bank of amplifiers for amplification.

21. The method of claim 12, wherein the composite structure is a mixture of water and a collagen structure.

22. The method of claim 21, wherein pulse modulated waveform vibrates water molecules near the collagen structure to produce heating.

23. A system for transferring radio frequency energy to a collagen structure comprising:
a radio signal generator that produces a pulse modulated waveform with variable carrier frequency and variable pulse repetition frequency, the pulse modulated waveform is applied to the collagen structure for heating;
an infrared imaging element that measures the rate of heating in the composite structure for particular values of the variable carrier frequency and the variable pulse repetition frequency, the infrared imaging element produces a representation illustrating the effects of heating on the collagen structure as well as the sample's molecular, nanoscopic structural, or chemical characteristics; and
a controller that determines the optimum variable center frequency and the variable pulse repetition frequency for optimum heating of the collagen structure while minimizing damage.

24. The system of claim 23, wherein the infrared imaging element comprises a thermal camera.

25. The system of claim 24, wherein the thermal camera illustrates regions of heating using color.

26. The system of claim 23, wherein the infrared imaging element comprises a Raman spectrometer.

27. The system of claim 26, wherein the Raman spectrometer illustrates position, shape and intensity of spectral lines characteristic of molecules.

28. The system of claim 23, wherein the controller determines the optimum carrier frequency and pulse repetition frequency by sending a control signal to the radio signal generator.

29. The system of claim 23, wherein the optimum carrier frequency and pulse repetition frequency are determined by the maximum difference in heating between regions subject to a constraint of a minimum degree of heating of the composite structure.

30. The system of claim 24, wherein the thermal camera incorporates magnification and focus to record an image on a digital focal plane to be transmitted to the image processor.

31. The system of claim 23, wherein the radio signal generator comprises a bank of oscillators that are coupled to a bank of amplifiers for amplification.

32. The system of claim 23, wherein the collagen structure is mixed with water.

33. The system of claim 32, wherein pulse modulated waveform vibrates water molecules near the collagen structure to produce heating.
